(19) United States
(12) Patent Application Publication
(10) Pub. No.: US 2014/0121523 A1
(43) Pub. Date: May 1, 2014

(54) MEASURING APPARATUS AND METHOD THEREOF

(71) Applicant: GE Medical Systems Global Technology Company, LLC, Waukesha, WI (US)

(72) Inventors: Seiji Funaya, Toyko (JP); Koji Miyama, Tokyo (JP); Yasuyo Saito, Tokyo (JP); Masafumi Ogasawara, Tokyo (JP)

(73) Assignee: GE MEDICAL SYSTEMS GLOBAL TECHNOLOGY COMPANY, LLC, Waukesha, WI (US)

(21) Appl. No.: 14/066,399

(22) Filed: Oct. 29, 2013

(30) Foreign Application Priority Data
Oct. 30, 2012 (JP) 2012-238921

(51) Int. Cl. A61B 8/08

(52) U.S. Cl. CPC A61B 8/0858
USPC 600/449

(57) ABSTRACT

A measuring apparatus is provided. The measuring apparatus includes a measurement value calculating unit configured to calculate a measurement value, based on each of a plurality of measurement reference points set to an ultrasound image that is generated based on echo signals of ultrasound obtained from a subject, an evaluation value calculating unit configured to calculate an evaluation value related to one of a rate of reflection of each of the echo signals and an azimuth of a biological tissue structure in the subject, the evaluation value calculated with respect to each of the measurement reference points, and a determination unit configured to determine a reliability for the measurement value, based on the evaluation value.

### CROSS REFERENCE TO RELATED APPLICATIONS

This application claims the benefit of Japanese Patent Application No. 2012-238921 filed Oct. 30, 2012, which is hereby incorporated by reference in its entirety.

### BACKGROUND OF THE INVENTION

The present invention relates to a measuring apparatus which measures a vascular diameter, a wall thickness of a vascular wall and the like, and a control program thereof.

Japanese Unexamined Patent Publication No. 2012-183261 discloses an ultrasound diagnostic apparatus which in order to calculate a coefficient of elasticity of a blood vessel or the like, tracks measurement reference points set to an ultrasound image to measure vascular wall thicknesses and the like.

Meanwhile, there is a case where the average value of wall thicknesses or vascular diameters at a plurality of points is calculated as a wall thickness of a blood vessel or a vascular diameter at a minor-axis section of the blood vessel. Here, within the vascular wall at the minor-axis section of the blood vessel, portions such as its upper and lower portions at each of which the angle to a sound ray direction of ultrasound is large, are large in the reflectivity of each echo signal. Therefore, the contrast between the vascular wall and each portion other than it is clear in an ultrasound image. It is thus possible to accurately track the motion of the vascular wall.

There is however a case where since portions at each of which the angle to the sound ray direction of the ultrasound is small within the vascular wall, are small in the reflectivity of each echo signal, the contrast between the vascular wall and each portion other than it is not clear in the ultrasound image, thus causing a difficulty in accurately tracking the motion of the vascular wall. Accordingly, there is a case where when the average value is calculated inclusive of the wall thickness and diameter of a portion low in the intensity of each echo signal, the accurate value cannot be obtained.

From the above, there has been a demand for a measuring apparatus capable of preventing a measurement value at each measurement reference point from becoming incorrect, and a control program thereof.

### BRIEF DESCRIPTION OF THE INVENTION

In one aspect, a measuring apparatus which is equipped with a measurement value calculating unit is provided. The measuring apparatus calculates a measurement value, based on each of measurement reference points set to an ultrasound image generated based on echo signals of ultrasound obtained from a subject, an evaluation value calculating unit which calculates an evaluation value related to each parameter that exerts an influence on a rate of reflection of each of the echo signals or its reflectivity, or an evaluation value related to an azimuth of a biological tissue structure in the subject with respect to each of the measurement reference points, and a determination unit which determines reliability for the measurement value calculated based on each of the measurement reference points, based on the evaluation value.

According to the above aspect, the reliability for each of the measurement values calculated at the measurement reference points is determined based on the evaluation value related to the rate of reflection of each of the echo signals or the evaluation value related to the azimuth of the biological tissue structure in the subject. It is therefore possible to prevent the measurement value from becoming incorrect by the influence of parameters that exert an influence on the rate of reflection of the echo signal or its reflectivity.

Further advantages will be apparent from the following description of exemplary embodiments as illustrated in the accompanying drawings.

### BRIEF DESCRIPTION OF THE DRAWINGS

FIG. 1 is a block diagram showing one example of a schematic configuration of an ultrasound diagnostic apparatus.

FIG. 2 is a block diagram of a function executed by a controller.

FIG. 3 is a diagram showing a B-mode image of a minor-axis section of a blood vessel displayed on a display unit.

FIG. 4 is a flowchart illustrating the operation of setting a first circle and a second circle.

FIG. 5 is an explanatory diagram of the setting of the first and second circles and a diagram showing a state in which three points are designated.

FIG. 6 is an explanatory diagram of the setting of the first and second circles and a diagram showing a state in which the first circle passing through the three points set in FIG. 5 is set.

FIG. 7 is an explanatory diagram of the setting of the first and second circles and a diagram for describing that the outline of a vascular outer wall is searched from the first circle set in FIG. 5.

FIG. 8 is an explanatory diagram of the setting of the first and second circles and a diagram showing a state in which the second circle is set.

FIG. 9 is a diagram for describing the calculation of an average value of distances between the first and second circles.

FIG. 10 is a diagram for describing a point comprised of an aggregate of a plurality of pixels.

FIG. 11 is a diagram for describing the calculation of an average value of inner diameters.

FIG. 12 is a diagram for describing the calculation of an average value of outer diameters.

FIG. 13 is a flowchart showing the operation of measurement.

FIG. 14 is a diagram for describing each part to determine an average value of a rate of change in brightness.

FIG. 15 is a diagram for describing that the rate of change in brightness at a point comprised of an aggregate of a plurality of pixels is calculated from some pixels.

FIG. 16 is a diagram for describing an angle which the direction of a biological tissue structure and the sound ray direction of ultrasound form with each other.

FIG. 17 is a diagram for describing an angle which the direction of the biological tissue structure and the direction perpendicular to the sound ray direction of ultrasound form with each other.

FIG. 18 is a diagram for describing the specification of the direction of a biological tissue structure.

FIG. 19 is a diagram for describing the specification of the direction of a biological tissue structure.

FIG. 20 is a diagram for describing the specification of the direction of a biological tissue structure.

FIG. 21 is a diagram for describing a range of ±θth1 to the sound ray direction of ultrasound.

FIG. 22 is a diagram for describing a range of ±θth2 to the direction perpendicular to the sound ray direction of ultrasound.

FIG. 23 is a diagram for describing the extraction of a vascular outline and a diagram showing a state in which two points at a vascular wall are designated.

FIG. 24 is a diagram for describing the extraction of the vascular outline and a diagram showing a state in which points on the outline of a vascular inner wall and points on the outline of a vascular outer wall are specified.

FIG. 25 is a diagram for describing the extraction of the vascular outline and a diagram for describing the extraction of the outlines of the vascular inner and outer walls.

FIG. 26 is a diagram for describing the extraction of the vascular outline and a diagram for describing that the outlines of the vascular inner and outer walls are extracted.

### DETAILED DESCRIPTION OF THE INVENTION

### First Embodiment

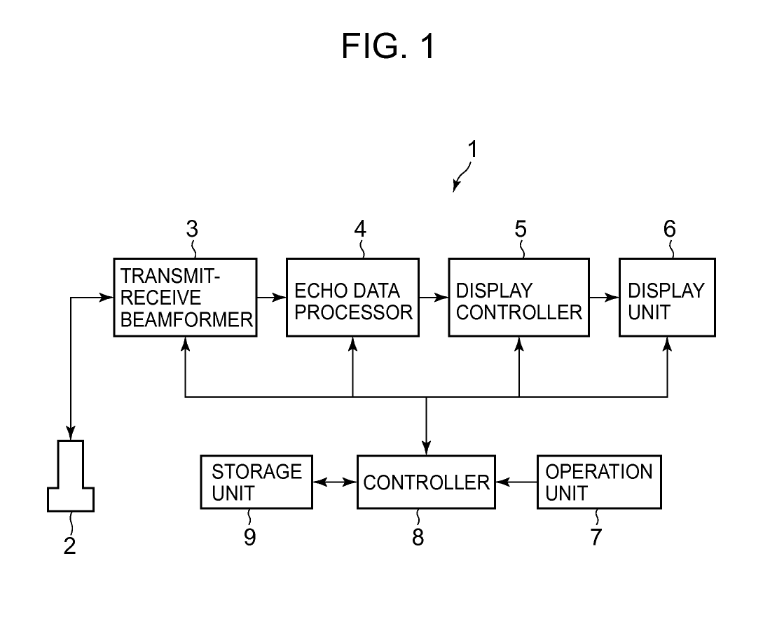

A first embodiment will hereinafter be described in detail based on FIGS. 1 through 14. An ultrasound diagnostic apparatus 1 shown in FIG. 1 is equipped with an ultrasound probe 2, a transmit-receive beamformer 3, an echo data processor 4, a display controller 5, a display unit 6, an operation unit 7, a controller 8 and a storage unit 9. The ultrasound diagnostic apparatus 1 is one example illustrative of an embodiment of a measuring apparatus.

The ultrasound probe 2 includes a plurality of ultrasound transducers (not shown) arranged in array form. The ultrasound probe 2 transmits ultrasound to a subject through the ultrasound transducers and receives its echo signals therein.

The transmit-receive beamformer 3 supplies an electric signal for transmitting ultrasound from the ultrasound probe 2 under a predetermined scan condition to the ultrasound probe 2, based on a control signal outputted from the controller 8. Also, the transmit-receive beamformer 3 performs signal processing such as A/D conversion, phasing-adding processing and the like on each echo signal received by the ultrasound probe 2 and outputs echo data subsequent to the signal processing to the echo data processor 4.

The echo data processor 4 performs signal processing for generating an ultrasound image on the echo data outputted from the transmit-receive beamformer 3. For example, the echo data processor 4 performs B-mode processing including logarithmic compression processing and envelope detection processing or the like to generate B-mode data.

The display controller 5 performs scan conversion based on a scan converter on the B-mode data to generate B-mode image data. The display controller 5 causes the display unit 6 to display a B-mode image based on the B-mode image data.

The display unit 6 an LCD (Liquid Crystal Display), a CRT (Cathode Ray Tube) or the like. The operation unit 7 includes a keyboard and a pointing device (not shown) or the like for inputting instructions and information by an operator.

The controller 8 is a CPU (Central Processing Unit) and reads a control program stored in the storage unit 9 to execute functions at the respective parts of the ultrasound diagnostic apparatus 1. For example, the functions of the transmit-receive beamformer 3, the echo data processor 4 and the display controller 5 may be executed by the control program.

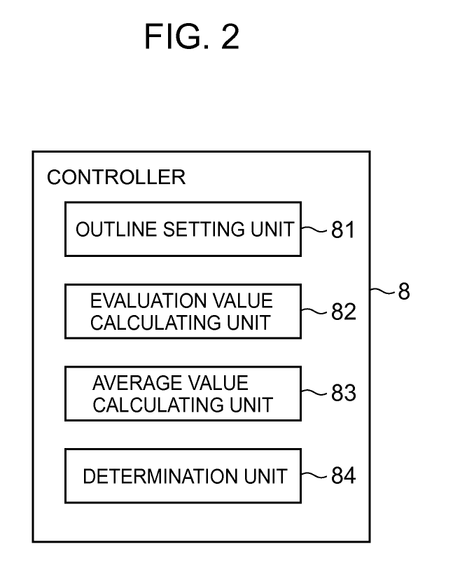

Further, the controller 8 cause the functions of an outline setting unit 81, an evaluation value calculating unit 82, an average value calculating unit 83 and a determination unit 84 shown in FIG. 2 to be executed. The details thereof will be described later.

The storage unit 9 is, for example, an HDD (Hard Disk Drive), a semiconductor memory or the like.

A description will now be made of the operation of the ultrasound diagnostic apparatus 1 according to the first embodiment. A description will be made here of the operation of measuring the blood vessel of a subject using the ultrasound diagnostic apparatus 1. The measurement for the blood vessel includes, for example, measurements of the thickness of a vascular wall and a vascular diameter at the minor-axis section of the blood vessel, etc. These measurements are carried out by tracking the motion of the vascular wall.

Upon execution of the measurement, the ultrasound probe 2 first performs the transmission/reception of ultrasound to and from the subject to obtain echo signals. The direction of the ultrasound probe 2 is assumed to be a direction in which the azimuth direction (the direction of arrangement of ultrasound transducers) of the ultrasound probe 2 is perpendicular to the travel direction of the blood vessel of the subject. Then, the echo data processor 4 generates B-mode data, based on the echo signals acquired by the ultrasound probe 2. The B-mode data is row data and stored in the storage unit 9. A real-time B-mode image may be displayed upon the transmission/reception of the ultrasound.

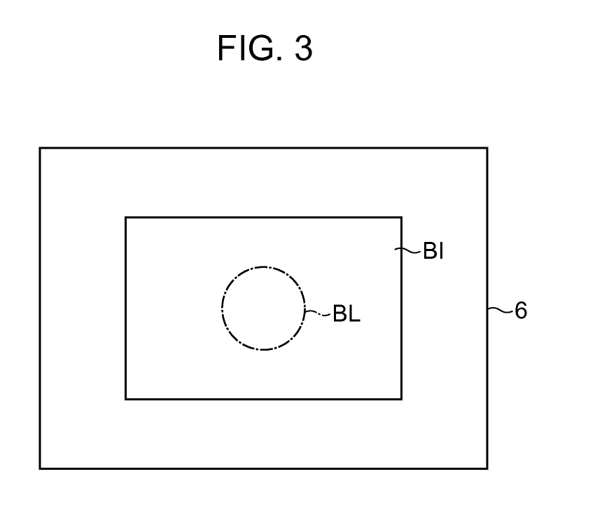

The measurement is performed after the completion of the transmission/reception of the ultrasound. Described specifically, B-mode data is first read from the storage unit 9. Then, the display controller 5 generates B-mode image data, based on the B-mode data and causes the display unit 6 to display a B-mode image BI as shown in FIG. 3. The B-mode image BI is an image of a minor-axis section of a blood vessel BL indicated by a one-dot chain line.

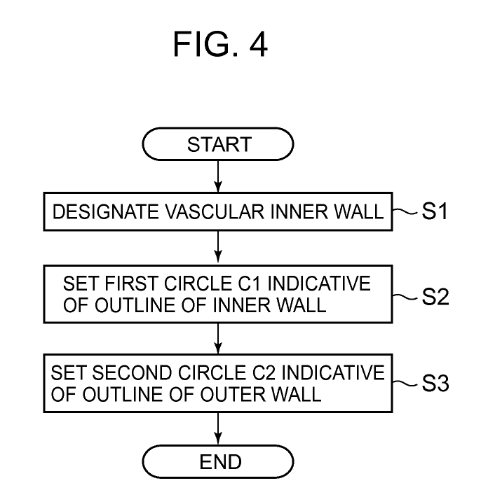

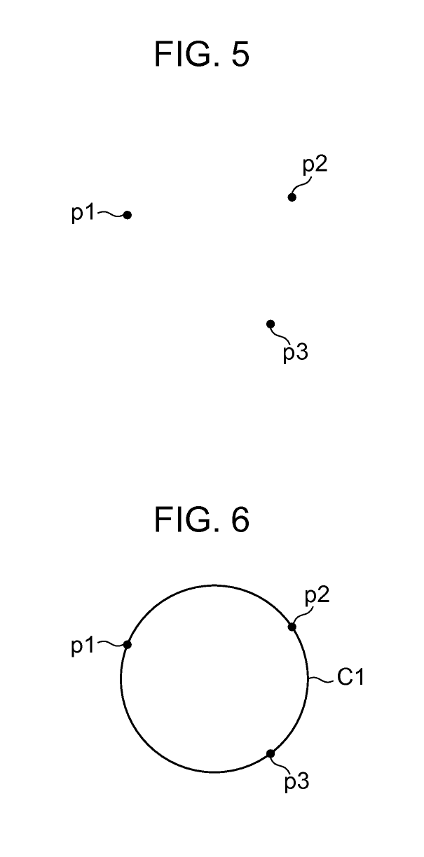

When the B-mode image BI is displayed on the display unit 6, a first circle C indicative of an inner wall of vascular walls of the blood vessel BL, and a second circle C2 indicative of an outer wall thereof are set. The setting of the first and second circles C1 and C2 is performed at the B-mode image BI corresponding to a still picture. This will specifically be explained based on the flowchart of FIG. 4. First, at Step S1, the operator designates three points p1, p2 and p3 of the inner wall of the blood vessel BL in the B-mode image BI as shown in FIG. 5 by a cursor (not shown) displayed on the display unit 6, using the operation unit 7. Incidentally, the blood vessel BL is not shown in FIG. 5.

Next, at Step S2, the outline setting unit 81 sets a first circle C1 passing through the points p1, p2 and p3 specified at Step S1 described above as shown in FIG. 6. This first circle C1 is a circle indicative of the outline of the inner wall of the blood vessel.

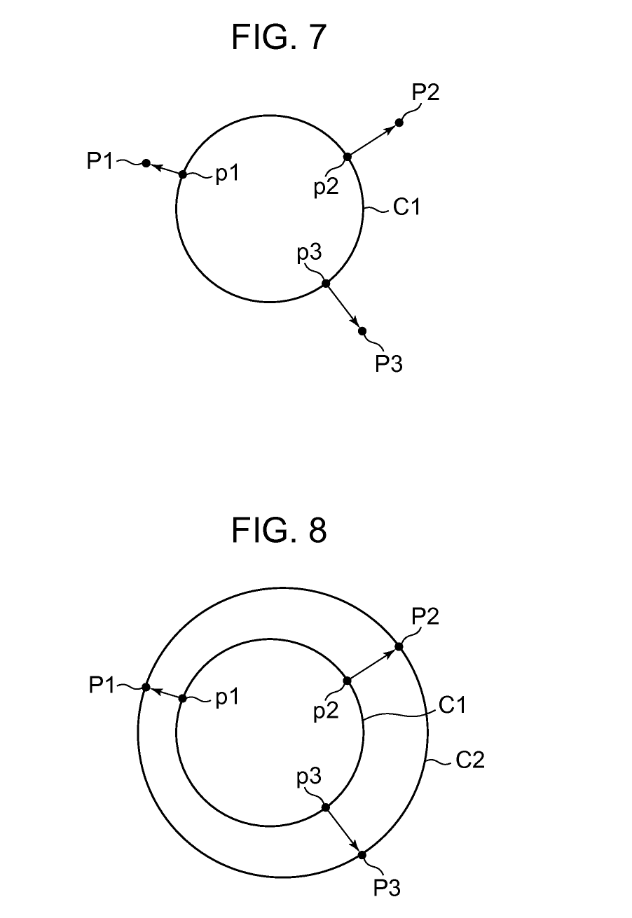

When the first circle C1 is set at Step S2, the outline setting unit 81 sets a second circle C2 larger in diameter than the first circle C1 to the outside of the first circle C1 at Step S3. For example, as shown in FIG. 7, the outline setting unit 81 searches for the outline of the outer wall of the blood vessel toward the outside of the first circle C1 from the points p1, p2 and p3 and thereby specifies points P1, P2 and P3 at which the difference in brightness of the B-mode image BI or the rate of change in its brightness becomes larger than a prescribed threshold value. Incidentally, the outline setting unit 81 calculates the difference in brightness or the rate of change in brightness, based on the intensity (data value of B-mode data) of each echo signal and thereby specifies the points P1, P2 and P3. When the points P1, P2 and P3 are specified, the outline setting unit 81 sets a second circle C2 that passes through the points P1, P2 and P3 as shown in FIG. 8. This second circle C2 is a circle indicative of the outline of the outer wall of the blood vessel.

The first circle C1 and the second circle C2 may or may not be displayed on the display unit 6.

Incidentally, the circles are set herein, but ellipses other than the circles may be set. That is, a first ellipse C1′ may be set instead of the first circle C1, and a second ellipse C2′ may be set instead of the second circle C2.

When the first circle C1 and the second circle C2 are set in the above-described manner, the measurement of the blood vessel BL is started. The measurement is performed based on the points on the first circle C1 and the second circle C2 (or the first ellipse C1′ and second ellipse C2′). The measurement is carried out at plural time phases by tracking the movements of the points on the first circle C1 and the second circle C2 (or the first ellipse C1′ and second ellipse C2′) with the pulsation of the blood vessel. The measurement is intended for the measurements of the wall thickness of a blood vessel, the diameter thereof and the like.

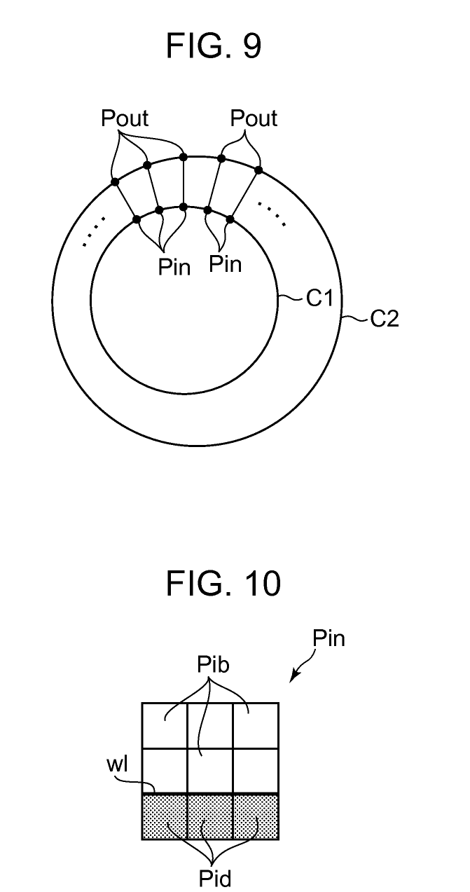

As the wall thickness, an average wall thickness Wav is calculated at each time phase. Described specifically, when first and second circles C1 and C2 shown in FIG. 9, for example, are not concentric circles, distances X between points Pin of the first circle C1 and points Pout of the second circle C2 are calculated plural. The average value of these plural distances X is calculated as an average wall thickness. Incidentally, in FIG. 9, the points Pin and Pout are illustrated only by five points at the first and second circles C1 and C2 respectively, and only some are illustrated.

The points Pin and Pout are points comprised of plural adjacent pixels. The points Pin and Pout are targeted for tracking movements in a B-mode image and are respectively an aggregate of a plurality of adjacent pixels pid and pib such as shown in FIG. 10, for example. As a method for tracking the movements of the points Pin and Pout, a known method such as an optical flow method or the like is used.

The point Pin related to the first circle C1 is illustrated in FIG. 10. Each of the pixels pid is a portion having dots in FIG. 10, which corresponds to the lumen of the blood vessel. Each of the pixels pib is a portion having no dots, which corresponds to the vascular wall. Thus, the outline of the inner wall is shown between the pixels pid and pib as indicated by a thick line w1 in the drawing. That is, the point Pin includes the outline of the inner wall.

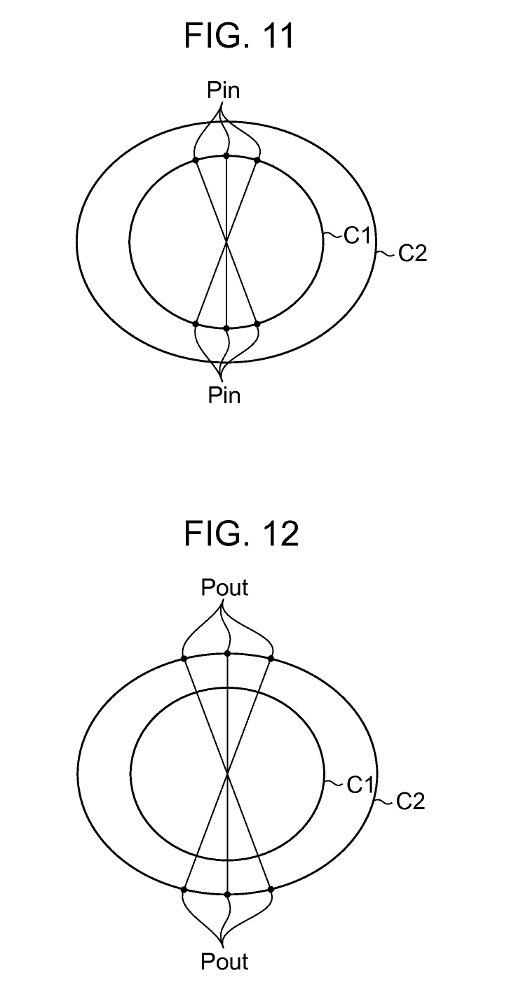

For example, when a first ellipse C1′ and a second ellipse C2′ other than the first circle C1 and the second circle C2 are set, an average vascular diameter Dav is calculated as a vascular diameter at each time phase. Both of the inner and outer diameters may be calculated as the vascular diameter. Specifically, as shown in FIG. 11, distances Yi between two points Pin and Pin on the first ellipse C1′ are calculated plural. The average value of the distances Yi is calculated as the average value of the inner diameter. As shown in FIG. 12, distances Yo between two points Pout and Pout on the second ellipse C2′ are calculated plural. The average value of the distances Yo is calculated as the average value of the outer diameter.

By the way, in FIGS. 11 and 12, for clarity the points Pin and Pout are illustrated using only six points, and only some are illustrated.

Here, since a portion at which the rate of reflection of each echo signal on the ultrasound probe 2 is small is in danger of causing a large measurement error, the measurement is carried out after the points not used in the calculation of the average wall thickness Wav and the average vascular diameter Dav have been specified. Incidentally, the measurement itself may not be performed on the points not used in the calculation of the average wall thickness Wav and the average vascular diameter Dav.

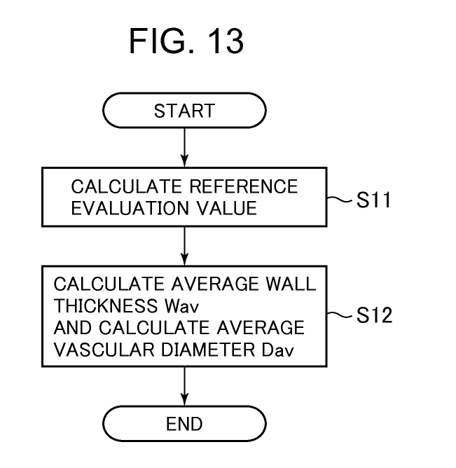

A flow for the measurement will specifically be described based on the flowchart of FIG. 13. First, at Step S11, the determination unit 84 calculates a reference evaluation value related to the rate of reflection of each echo signal onto the ultrasound probe 2 as a reference for specifying each of the points not used in the calculation of the average wall thickness Wav and the average vascular diameter Dav.

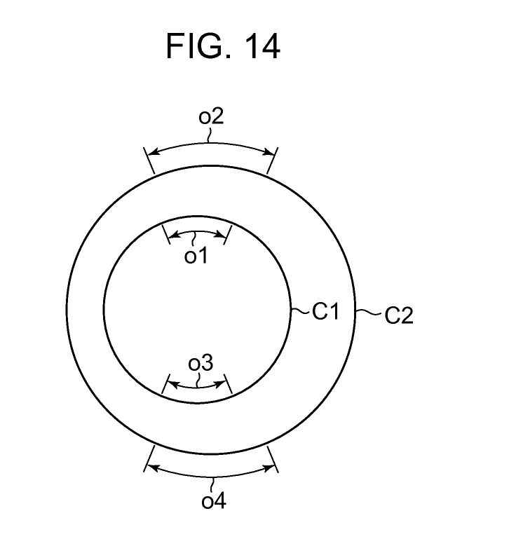

The calculation of the reference evaluation value will be described in detail. First, the determination unit 84 calculates an average value Bray of rates of change in the brightness Br (to be described later) of the pixels pid and pib with respect to all points Pin and Pout at parts o1, o2, o3 and o4 of the first and second circles C1 and C2 shown in FIG. 14 (first and second ellipses C1′ and C2′ are also similar). The parts o1, o2, o3 and o4 of the first and second circles C1 and C2 are portions that are located above and below the vascular wall and have angles perpendicular to the sound ray direction of ultrasound or near perpendicular thereto. Thus, at the portions having the angles relatively large with respect to the sound ray direction of the ultrasound, the rate of reflection of each echo signal on the ultrasound probe 2 is large and the contrast between the vascular wall and each portion other than it is clear. The points Pin and Pout at the parts o1, o2, o3 and o4 of the first and second circles C1 and C2 are one example illustrative of an embodiment of prescribed measurement reference points set in advance as being large in the rate of reflection of each echo signal.

Each of the parts o1, o2, o3 and o4 of the first and second circles C1 and C2 may be one section obtained by equally dividing the circumstance of each circle in plural form, for example.

The rate of change in the brightness Br of each point Pin and Pout is calculated based on the average of brightness of all pixels pid and the average of brightness of all pixels pib at the respective points Pin and Pout. Here, for example, the pixels pid are pixels each having brightness larger than a prescribed threshold value. The pixels pib are pixels each having brightness below a prescribed threshold value.

Next, the determination unit 84 calculates a reference evaluation value E by the following Equation 1:

E=k×Brav Equation 1

where k<1. k is set to a value at which the rate of change in brightness that makes it possible to track the vascular wall as correct as possible, can be obtained as the reference evaluation value E.

Next, at Step S12, the average value calculating unit 83 calculates an average wall thickness Wav and an average vascular diameter Dav. Upon their calculation, the determination unit 84 compares the rate of change in brightness Br at each of the points Pin and Pout and the reference evaluation value E and determines reliability with respect to the measurement values (the distances X, Yi and Yo) calculated based on the respective points Pin and Pout. Specifically, the determination unit 84 determines that the measurement values calculated based on the respective points Pin and Pout defined as being E≦Br are high in reliability and can be used as the measurement values to calculate the average wall thickness Wav and the average vascular diameter Day. Upon determination by the determination unit 84, however, the measurement values may or may not be calculated.

The average value calculating unit 83 calculates the distances X, Yi and Yo using only the points Pin and Pout defined as being E≦Br specified by the determination unit 84, and calculates the average wall thickness Wav and the average vascular diameter Dav. Thus, the points Pin and Pout defined as being E>Br are eliminated and the average wall thickness Wav and the average vascular diameter Dav are calculated.

If the rate of change in brightness Br at at least any of the points Pin and Pout for calculating the distances X is smaller than the reference evaluation value E, the distance X at that time is not included in the distances for calculating the average wall thickness Wav. If the rate of change in brightness Br at at least any of the points Pin1 and Pin2 for calculating the distances Yi is smaller than the reference evaluation value E, the distance Yi at that time is not included in the distances for calculating the average value of the inner diameter. Further, if the rate of change in brightness Br at at least any of the points Pout1 and Pout2 for calculating the distances Yo is smaller than the reference evaluation value E, the distance Yo at that time is not included in the distances for calculating the average value of the outer diameter.

The rate of change in brightness Br at each of the points Pin and Pout is calculated by the evaluation value calculating unit 82.

The average value calculating unit 83 tracks the movements of the points Pin and Pout with the pulsation of the blood vessel to thereby calculate the average wall thickness Wav and the average vascular diameter Dav and calculate the rate of change or the like with respect to, for example, a blood vessel having the minimum diameter and a blood vessel having the maximum diameter respectively.

According to the first embodiment, the average wall thickness Wav and the average vascular diameter Dav are calculated without including the distances X, Yi and Yo calculated from the points Pin and Pout where the rate of change in brightness Br is smaller than the reference evaluation value E. It is therefore possible to prevent the calculated average values from becoming incorrect due to the influence of the rate of reflection of each echo signal.

A modification will next be explained. Upon the calculation of the reference evaluation value E, the determination unit 84 may calculate the average value Bra of the rates of change in the brightness Br with respect to some plural points Pin and Pout without including all points Pin and Pout at the parts o1, o2, o3 and o4 of the first circle C1 and the second circle C2 (or first ellipse C1′ and second ellipse C2′). Also the determination unit 84 may calculate the average value Bray with respect to any one of the parts o1, o2, o3 and o4 of the first circle C1 and the second circle C2 (or first ellipse C1′ and second ellipse C2′), or all of the plural parts or some plural points Pin and Pout. That is, the determination unit 84 may calculate the average value Bray with respect to, for example, all points Pin or some plural points Pin at the part o1 of the first circle C1. Also, the determination unit 84 may calculate the average value Bray with respect to, for example, all points Pin or some plural points Pin at the parts o1 and o3 of the first circle C1.

Further, the determination unit 84 may calculate the reference evaluation value E by the following Equation 2:

E=k×Br Equation 2

That is, the determination unit 84 may multiply the rate of change in the brightness Br about one point of the points pin and Pout at any of the parts o1, o2, o3 and o4 of the first circle C1 and the second circuit C2 (or first ellipse C1′ and second ellipse C2′) by the coefficient k (k<1) to calculate the reference evaluation value E.

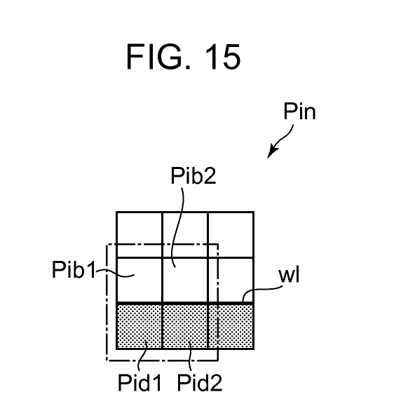

Further, the rate of change in the brightness Br at each of the points Pin and Pout may be calculated from some pixels at each of the points Pin and Pout. As shown in FIG. 15, for example, the rate of change in brightness at the point Pin may be calculated based on the average of some pixels pid1 and pid2 surrounded by a one-dot chain line, and the average of brightness of some pixels pib1 and pib2.

### Second Embodiment

A second embodiment will next be described. A block configuration of an ultrasound diagnostic apparatus 1 according to the second embodiment is similar to the first embodiment, and its description will therefore be omitted.

The operation of the ultrasound diagnostic apparatus 1 according to the second embodiment will be described. The second embodiment is similar to the first embodiment except for a flow for measurement. The measurement flow in the second embodiment is different from the flow in the first embodiment shown in FIG. 13, and a reference evaluation value has been set. The reference evaluation value is different from the first embodiment and will be described later.

A description will be made of the calculation of an average wall thickness Wav and the calculation of an average vascular diameter Dav in the second embodiment. Before the calculation of the average wall thickness Wav and the average vascular diameter Dav, the evaluation value calculating unit 82 calculates an angle θ to a base line bl as viewed in the direction bt of a biological tissue structure with respect to the points Pin and Pout.

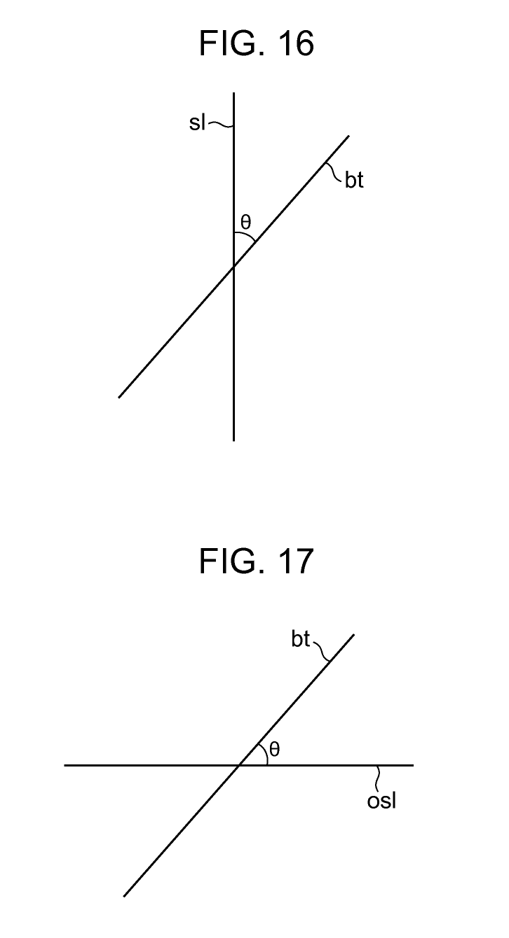

For example, the base line bl may be a sound ray direction sl of ultrasound as shown in FIG. 16. An angle θ which the sound ray direction sl of the ultrasound and the direction bt of the biological tissue structure form with each other may be calculated. Further, as shown in FIG. 17, the base line bl may be a direction osl perpendicular to the sound ray direction of the ultrasound. Alternatively, an angle θ which the orthogonal direction osl and the direction bt of the biological tissue structure form with each other may be calculated.

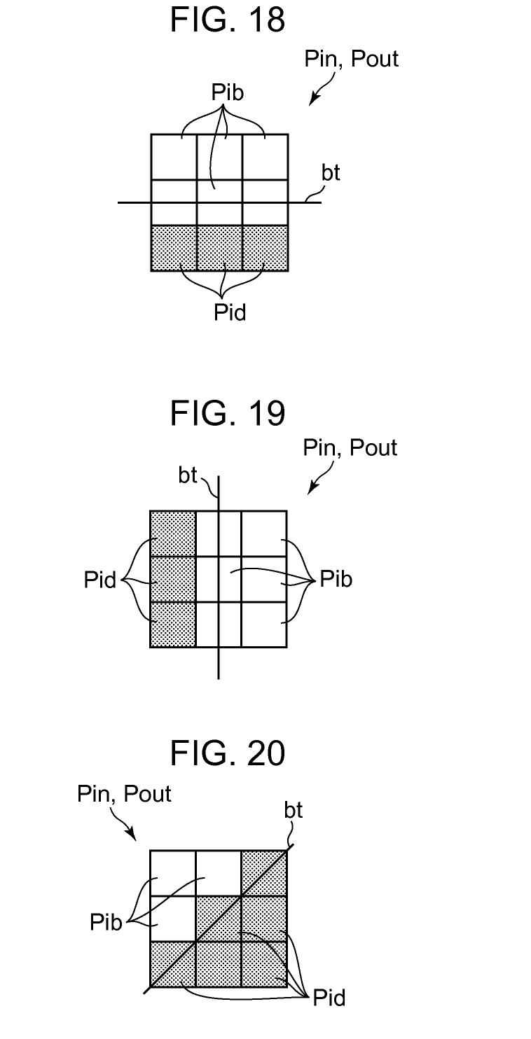

The direction bt of the biological tissue structure is the direction of a vascular wall herein. The direction bt of the biological tissue structure is specified from a spatial intensity distribution (brightness distribution) of echo signals at an ultrasound image such as a B-mode image. Specifically, a brightness gradient vector is calculated based on a spatial brightness distribution at points Pin and Pout to specify the direction bt. When pixels pid and pib are arranged in a horizontal direction at a point Pin or Pout as shown in FIG. 18, for example, the horizontal direction is assumed to be the direction bt of the biological tissue structure. When pixels pid and pib are arranged in a vertical direction at a point Pin or Pout as shown in FIG. 19, the vertical direction is assumed to be the direction bt of the biological tissue structure. When pixels pid and pib are arranged in an oblique line at a point Pin or Pout as shown in FIG. 20, the oblique direction is assumed to be the direction bt of the biological tissue structure.

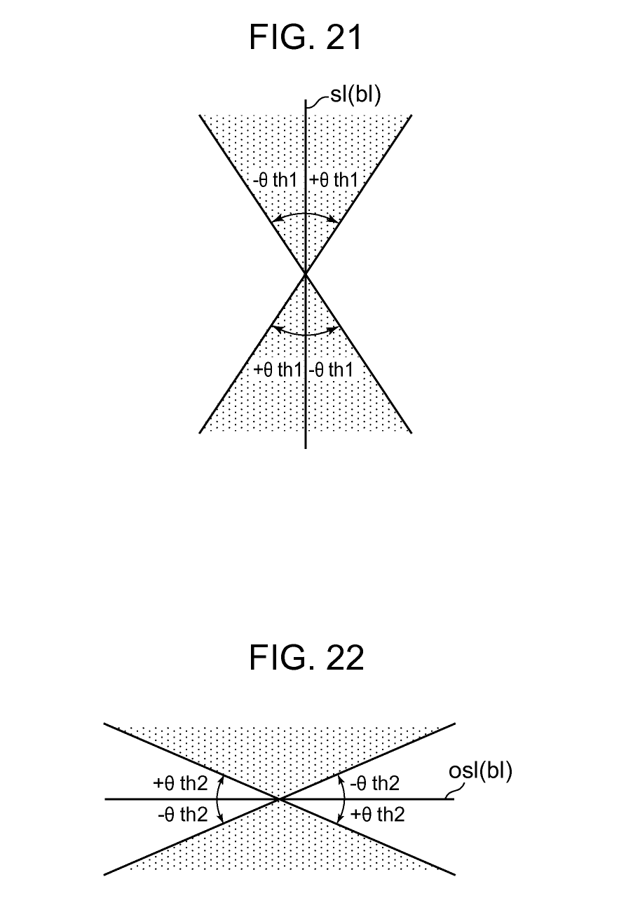

Upon calculation of the average wall thickness Wav and the average vascular diameter Dav by the average value calculating unit 83, the determination unit 84 compares the angle θ at each of the points Pin and Pout and a reference evaluation value E′ to determine reliability. The reference evaluation value E′ used herein is an angle set with respect to the base line bl. If, for example, the angle θ at each point Pin, Pout is within a range of ±θth1 with respect to the sound ray direction sl of ultrasound being the base line bl as shown in FIG. 21 (if it is within a range shown in dots in FIG. 21), the determination unit 84 determines that measurement values (the distances X, Yi and Yo) calculated based on the points Pin and Pout are high in reliability. Here, ±θth1 is the reference evaluation value E′.

When the angle θ calculated at each of the points Pin and Pout is not within a ±θth2 with respect to a direction osl perpendicular to the sound ray direction of ultrasound being a base line bl as shown in FIG. 22, for example (if it is within a range shown in dots in FIG. 22), the determination unit 84 determines that measurement values (the distances X, Yi and Yo) calculated at the points Pin and Pout are high in reliability. Here, ±θth2 is the reference evaluation value E′.

The ±θth1 and ±θth2 may be stored in defaults or set by the operator. The ±θth1 and ±θth2 are set in terms of whether the rate of reflection of each echo signal can be ensured and the tracking of the vascular wall can be done as correct as possible.

The average value calculating unit 83 uses the points Pin and Pout determined to be within the ranges of the ±θth1 and ±θth2 by the determination unit 84 in the calculation of the average wall thickness Wav and the average vascular wall Day.

According to the second embodiment, the average wall thickness Wav and the average vascular diameter Dav are calculated using the points Pin and Pout at the portions each having the angle at which the motion of the vascular wall can be done as correct as possible. It is therefore possible to prevent the calculated average values from becoming incorrect due to the influence of the rate of reflection of each echo signal.

Although the disclosure has been described above by the respective exemplary embodiments, it is needless to say that the systems and methods described herein can be changed in various ways within the scope of and without changing the spirit of the invention. For example, since the actual outline of vascular wall may differ from each of the first and second circles C1 and C2, the outline setting unit 81 may search for the outline of a vascular wall in a B-mode image with points in each of the first and second circles C1 and C2 as search starting points and extract it. The outline of the vascular wall extracted with each point in the first circle C1 as the search starting point corresponds to the outline of the inner wall of the blood vessel. The outline of the vascular wall extracted with each point in the second circle C2 as the search starting point corresponds to the outline of the outer wall of the blood vessel. In this case, the calculation of the average wall thickness Wav and the average vascular diameter Day or the like is performed as with the above exemplary embodiments using the points on the outline of the vascular inner wall and the points on the outline of the vascular outer wall both extracted in the above-described manner as measurement reference points.

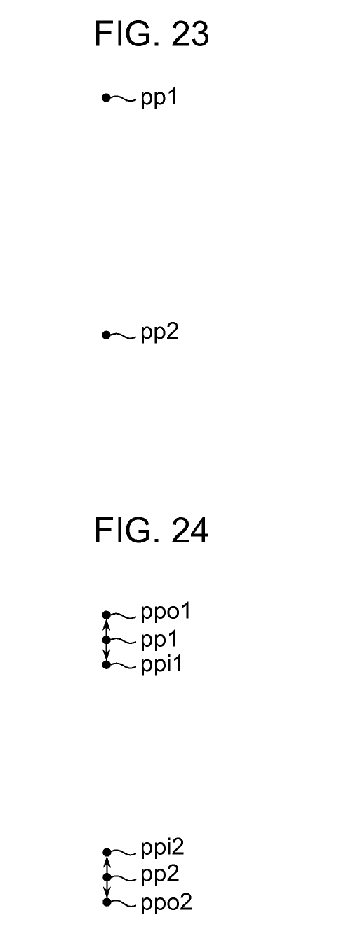

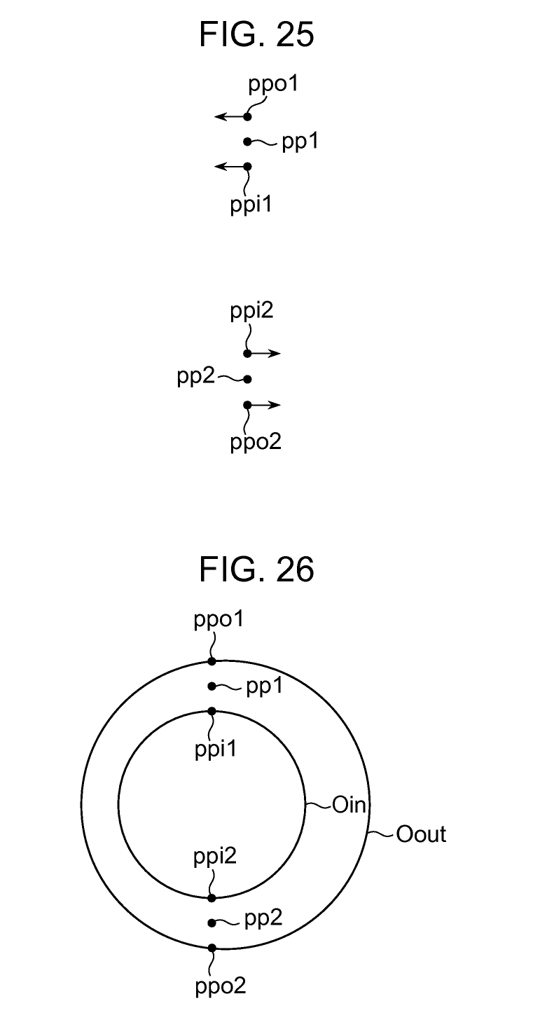

The extraction of the vascular outlines may be performed in the following manner without setting the first and second circles C1 and C2. That is, first, the operator designates a plurality of arbitrary points of a vascular wall on a B-mode image by a cursor or the like as shown in FIG. 23. In FIG. 23, two points of pp1 and pp2 have been designated. The outline setting unit 81 searches for inner and outer walls toward the directions of the inner and outer sides with the points pp1 and pp2 as search starting points. The outline setting unit 81 specifies points ppi1 and ppi2 on the outline of the inner wall and points ppo1 and ppo2 on the outline of the outer wall, based on the difference in brightness and the rate of change in brightness as shown in FIG. 24.

Next, the outline setting unit 81 searches for the inner and outer walls in the directions indicated by arrows, for example, with the points ppi1 and ppi2 on the inner wall and the points ppo1 and ppo2 on the outer wall as search starting points to thereby extract outlines. The outline setting unit 81 extracts the outline Oin of the inner wall and the outline Oout of the outer wall, based on the rate of change in brightness of the B-mode image as shown in FIG. 26. Incidentally, the outline Oin of the inner wall and the outline Oout of the outer wall are shown in perfect circles respectively in FIG. 26 but not limited thereto.

Incidentally, the method of extracting the outline Oin of the inner wall and the outline Oout of the outer wall is not limited to the above. For example, the operator designates a point to be considered as the outline of the inner wall on the B-mode image and a point to be considered as the outline of the outer wall thereon by the cursor or the like. Then, with the designated points as search starting points, the outline setting unit 81 may search for the outline of the inner wall and the outer wall in the B-mode image to extract their outlines.

In the respective exemplary embodiments described above, the difference in brightness (amount of change in brightness) may be used instead of the rate of change in brightness.

In the respective exemplary embodiments, the intensity (brightness of ultrasound image) of each echo signal may be used as an evaluation value instead of the rate of change in brightness and the difference in brightness. In this case, the evaluation value calculating unit 82 calculates the average value (average value of brightness) of the intensities of echo signals at pixels that configure the points Pin and Pout, for example, as the evaluation value. Also the evaluation value calculating unit 82 performs an average arithmetic operation on the average value of the intensities of echo signals of all (some) points Pin and Pout at the parts o1, o2, o3 and o4. Then, the evaluation value calculating unit 82 calculates the reference evaluation value E using the value obtained by the average arithmetic operation instead of the average value Bray of the rate of change in brightness Br in the above-described Equation 1. The average value calculating unit 83 compares the reference evaluation value E calculated in this manner and the average value of the intensities of the echo signals at the points Pin and Pout and calculates the average wall thickness Wav and the average vascular diameter Dav using Pin and Pout where the average value thereof is greater than or equal to the evaluation value E.

Although each of the above embodiments has explained where the average wall thickness Wav and the average vascular diameter Dav are calculated as the measurement values, the average value is not necessarily required to be calculated as the measurement value. For example, the results of tracking of the points Pin and Pout determined to be high in reliability by the determination unit 84 may be displayed on the display unit 6 as described in Japanese Unexamined Patent Publication No. 2012-90821. The distance between the points Pin and Pout determined to be high in reliability by the determination unit 84 may be displayed by one on the display unit 6 as described in Japanese Unexamined Patent Publication No. 2012-90821.

Further, although the measuring apparatus is realized for the ultrasound diagnostic apparatus in the exemplary embodiments described herein, the measuring apparatus may be implemented with respect to an apparatus other than the ultrasound diagnostic apparatus. For example, the measuring apparatus described herein may be implemented with respect to a general-purpose computer such as a personal computer. In this case, raw data such as B-mode data or image data such as B-mode image data is fetched into, for example, a general-purpose computer from the ultrasound diagnostic apparatus, and the processing described in the above exemplary embodiments is carried out by this general-purpose computer.

Many widely different embodiments may be configured without departing from the spirit and the scope of the present invention. It should be understood that the present invention is not limited to the specific embodiments described in the specification, except as defined in the appended claims.

1. A measuring apparatus comprising:
a measurement value calculating unit configured to calculate a measurement value, based on each of a plurality of measurement reference points set to an ultrasound image that is generated based on echo signals of ultrasound obtained from a subject;
an evaluation value calculating unit configured to calculate an evaluation value related to one of a rate of reflection of each of the echo signals and an azimuth of a biological tissue structure in the subject, the evaluation value calculated with respect to each of the measurement reference points; and
a determination unit configured to determine a reliability for the measurement value, based on the evaluation value.

2. The measuring apparatus of claim 1, wherein the evaluation value calculating unit is configured to calculate as the evaluation value, a value related to the intensity of each of the echo signals at the measurement reference points or a change in the echo signals.

3. The measuring apparatus of claim 2, wherein the evaluation value calculating unit is configured to calculate as the evaluation value, a rate of change in the intensity of the echo signal or an amount of change in the intensity.

4. The measuring apparatus of claim 1, wherein the evaluation value calculating unit is configured to calculate as the evaluation value, an angle which the direction of the biological tissue structure specified by the ultrasound image and a prescribed reference direction form with each other.

5. The measuring apparatus of claim 1, wherein the determination unit is configured to determine a reference evaluation value and the evaluation value at each of the measurement reference points by comparison therebetween.

6. The measuring apparatus of claim 2, wherein the determination unit is configured to determine a reference evaluation value and the evaluation value at each of the measurement reference points by comparison therebetween.

7. The measuring apparatus of claim 3, wherein the determination unit is configured to determine a reference evaluation value and the evaluation value at each of the measurement reference points by comparison therebetween.

8. The measuring apparatus of claim 4, wherein the determination unit is configured to determine a reference evaluation value and the evaluation value at each of the measurement reference points by comparison therebetween.

9. The measuring apparatus of claim 5, wherein the reference evaluation value is a value calculated by multiplying the intensity of an echo signal at a prescribed measurement reference point of the measurement reference points, or a rate of change in the intensity thereof or an amount of change in the intensity thereof by a prescribed coefficient.

10. The measuring apparatus of claim 6, wherein the reference evaluation value is a value calculated by multiplying an intensity of an echo signal at a prescribed measurement reference point of the plurality of measurement reference points, or by multiplying a rate of change in the intensity thereof or an amount of change in the intensity thereof by a prescribed coefficient.

11. The measuring apparatus of claim 7, wherein the reference evaluation value is a value calculated by multiplying an intensity of an echo signal at a prescribed measurement reference point of the plurality of measurement reference points, or by multiplying a rate of change in the intensity thereof or an amount of change in the intensity thereof by a prescribed coefficient.

12. The measuring apparatus of claim 9, wherein the prescribed measurement reference point is a measurement reference point set in advance as being large in the rate of reflection of the echo signal.

13. The measuring apparatus of claim 5, wherein the reference evaluation value is a prescribed angle set with respect to a prescribed reference direction.

14. The measuring apparatus of claim 1, including a display unit configured to display thereon a measurement value calculated based on each measurement reference point determined to be high in reliability by the determination unit.

15. The measuring apparatus of claim 1, wherein the measurement value calculating unit is configured to calculate a reflection value on which measurement values of measurement reference points determined to be high in reliability by the determination unit are reflected.

16. The measuring apparatus of claim 15, wherein the reflection value is an average value of the measurement values of the measurement reference points determined to be high in reliability.

17. The measuring apparatus of claim 1, wherein the measurement value calculating unit is configured to calculate measurement values targeted for a blood vessel of the subject.

18. The measuring apparatus of claim 17, wherein each of the measurement values targeted for the blood vessel is a value at a minor-axis section of the blood vessel.

19. The measuring apparatus of claim 18, wherein the measurement values targeted for the blood vessel are vascular diameters or wall thicknesses of vascular walls at plural points of the blood vessel.

20. A method of measuring a value comprising:
calculating a measurement value, based on each of a plurality of measurement reference points set to an ultrasound image that is generated based on echo signals of ultrasound obtained from a subject;
calculating an evaluation value related to one of a rate of reflection of each of the echo signals and an azimuth of a biological tissue structure in the subject, the evaluation value calculated with respect to each of the measurement reference points; and
determining reliability for the measurement value, based on the evaluation value.
